(19) United States
(12) Patent Application Publication
(10) Pub. No.: US 2026/0294767 A1
(43) Pub. Date: Oct. 1, 2026

(54) USE OF ERGOTHIONEINE IN PROMOTING COLLAGEN GENERATION

(71) Applicants: BLOOMAGE BIOTECHNOLOGY CORPORATION LIMITED, Shandong (CN); BEIJING BLOOMAGE HYINGC AND TECHNOLOGY CO., LTD, Beijing (CN); BLOOMATURE BIOTECHNOLOGY CO., LTD, Beijing (CN)

(72) Inventors: Shaoying LIU, Shandong (CN); Ruiyan WANG, Shandong (CN); Zhen LU, Shandong (CN); Yiming ZHANG, Shandong (CN); Xiaoou ZHANG, Shandong (CN); Hua MAO, Shandong (CN); Wenwen TAO, Shandong (CN)

(73) Assignees: BLOOMAGE BIOTECHNOLOGY CORPORATION LIMITED, Shandong (CN); BEIJING BLOOMAGE HYINGC AND TECHNOLOGY CO., LTD, Beijing (CN); BLOOMATURE BIOTECHNOLOGY CO., LTD, Beijing (CN)

(21) Appl. No.: 18/996,508

(22) PCT Filed: Jul. 25, 2023
(86) PCT No.: PCT/CN2023/109052

§ 371 Date: Jan. 17, 2025

(30) Foreign Application Priority Data
Jul. 26, 2022 (CN) 202210886121.5

(51) Int. Cl. A61K 8/49; A61K 31/4172; A61P 17/00; A61Q 19/08

(52) U.S. Cl. CPC A61K 8/4946; A61K 31/4172; A61P 17/00; A61Q 19/08

(57) ABSTRACT

The present application provides a collagen generation promoter comprising ergothioneine as an effective component, use of ergothioneine in promoting type III collagen generation, and use of ergothioneine in preparing a product for combating skin aging or preventing or alleviating skin problems related to type III collagen reduction. Ergothioneine has the effect of promoting type III collagen generation. Thus, as a collagen generation promoter, ergothioneine can be widely used for preparing products for promoting type III collagen generation and products for combating skin aging.

### TECHNICAL FIELD

The present application belongs to the field of cosmetics and food technology, and specifically, relates to use of ergothioneine in promoting collagen generation.

### BACKGROUND

Collagen is a biological macromolecular protein synthesized by animal cells. It is widely present in animals, abundant in content and diverse in variety, and has a significant impact on the normal function and injury repair of cells, tissues and even organs.

The collagen in adult skin tissue is mainly type I and type III collagen. In contrast, the proportion of type III collagen in fetal skin is significantly higher than that in adults. Reduction in type III collagen is associated with skin aging, resulting in a lack of elasticity and regenerative capacity. Supplementing type III collagen can reduce scar hyperplasia, promote skin regeneration, and accelerate wound healing. Human type III collagen can effectively promote macrophage transition from M1 to M2 phenotype, enhance L929 cell migration capacity, and stimulate angiogenesis in human umbilical vein endothelial cells (HUVECs).

However, the generation of type I and type III collagen is not positively correlated. Promoting the generation of type I collagen does not necessarily mean promoting the generation of type III collagen, and vice versa. Most of the related researches focus on the generation of type I collagen, with limited reports on how to promote the generation of type III collagen.

Ergothioneine (EGT) is a natural antioxidant that can protect cells in human body. It is a crucial active substance in the body and can be used in fields such as food, biomedicine, and cosmetics, etc. Although ergothioneine has been widely used, its efficacy remains not fully elucidated, and developing new applications of ergothioneine remains an urgent subject for research and resolution.

### SUMMARY

In view of the problems existing in the prior art, the present application unexpectedly discovered that ergothioneine has the effect of promoting the generation of type III collagen, thereby completing the present invention.

Specifically, this application involves the following aspects:

The present application provides a collagen generation promoter, comprising ergothioneine as an effective ingredient.

Furthermore, the collagen comprises type III collagen.

Furthermore, the collagen generation promoter is administered in vivo and/or in vitro.

The present application provides use of ergothioneine in promoting the generation of type III collagen.

Furthermore, the ergothioneine is administered in vivo and/or in vitro.

The present application provides use of ergothioneine in preparing a product for promoting the generation of type III collagen in skin.

The present application also provides use of ergothioneine in preparing a product for preventing or alleviating skin issues associated with the reduction of type III collagen.

Furthermore, the product comprises medicine, medical device, cosmetic or food.

The present application also provides a method for preventing or alleviating skin issues associated with the reduction of type III collagen, comprising administering ergothioneine to a subject in need thereof.

The present application also provides use of ergothioneine in preventing or alleviating skin issues associated with the reduction of type III collagen.

The present application discovered that ergothioneine has the effect of promoting the generation of type III collagen. Therefore, ergothioneine can be widely utilized as a collagen generation promoter in the preparation of products for promoting type III collagen generation and resisting skin aging.

### DETAILED DESCRIPTION

The present application is further described below in conjunction with embodiments. It should be understood that the embodiments are only used to further describe and illustrate the present application and are not used to limit the present application.

Unless otherwise defined, technical and scientific terms used in this specification have the same meanings as commonly understood by those skilled in the art. Although similar or identical methods and materials can be applied in experimental or practical applications, the materials and methods are still described below. In case of conflict, the present specification, including definitions, shall prevail. Additionally, the materials, methods, and embodiments are for illustrative purposes only and are not restrictive. The present application is further described below in conjunction with specific embodiments, but is not intended to limit the scope of the present application.

In view of the problems existing in the prior art, the inventors of the present application found that ergothioneine has the effect of promoting the generation of type III collagen, therefore a collagen generation promoter is provided, which comprises ergothioneine as an effective ingredient.

Those skilled in the art will appreciate that the collagen generation promoter may use ergothioneine as the sole active ingredient to promote the generation of type III collagen, or may contain other active ingredients that jointly promote the generation of type III collagen with ergothioneine.

In some embodiments, the administration method of the collagen generation promoter comprises in vivo and/or in vitro administration. When administered in vitro, for example, such as by smearing or applying externally, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered in vivo, such as by oral administration, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered by injection, the concentration of ergothioneine can be 10 μg/L-50 g/L, such as 10 μg/L, 100 μg/L, 1000 μg/L, 1 g/L, 10 g/L, 20 g/L, 30 g/L, 40 g/L, or 50 g/L etc., and any range between these values.

In some embodiments, the aforementioned collagen generation promoter can be administered to the skin directly or indirectly, for example, by smearing, applying externally, injection or oral administration.

In some embodiments, the aforementioned collagen generation promoter can be formulated into cosmetics, medical devices, medicines, foods, etc. For example, the aforementioned collagen generation promoter can be formulated alone or in combination with other active ingredients into cosmetics, medical devices, medicines, foods, etc., to promote the generation of type III collagen.

The present application also provides use of ergothioneine in promoting the generation of type III collagen. The use of ergothioneine in promoting the generation of type III collagen can be therapeutic or for non-therapeutic, such as cosmetic purposes.

In some embodiments, the administration method of the ergothioneine comprises in vivo and/or in vitro. When administered in vitro, for example, when smearing or applying externally, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered in vivo, such as by oral administration, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered by injection, the concentration of ergothioneine can be 10 μg/L-50 g/L, such as 10 μg/L, 100 μg/L, 1000 μg/L, 1 g/L, 10 g/L, 20 g/L, 30 g/L, 40 g/L, or 50 g/L etc., and any range between these values.

In the use of promoting type III collagen generation in the present application, ergothioneine can be administered to the skin directly or indirectly, for example, by smearing, applying externally, injection or oral administration.

In the use of promoting type III collagen generation in the present application, ergothioneine can be formulated into cosmetics, medical devices, medicines, foods, etc. For example, ergothioneine can be formulated alone or in combination with other active ingredients into cosmetics, medical devices, medicines, foods, etc., to promote the generation of type III collagen.

Furthermore, ergothioneine resists skin aging by promoting the generation of type III collagen.

In some embodiments, the administration method of the ergothioneine comprises in vivo and/or in vitro. When administered in vitro, for example, when smearing or applying externally, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered in vivo, such as by oral administration, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered by injection, the concentration of ergothioneine can be 10 μg/L-50 g/L, such as 10 μg/L, 100 μg/L, 1000 μg/L, 1 g/L, 10 g/L, 20 g/L, 30 g/L, 40 g/L, or 50 g/L etc., and any range between these values.

The present application also provides use of ergothioneine in preventing or alleviating skin issues associated with reduction of type III collagen.

The present application also provides a method for preventing or alleviating skin issues associated with type III collagen reduction, comprising administering ergothioneine to a subject in need thereof.

The present application also provides use of ergothioneine in preparing a product for preventing or alleviating skin issues associated with the reduction of type III collagen.

Wherein, skin issues associated with reduction of type III collagen can be various types of skin issues caused by reduction of type III collagen known in the art, such as decreased skin elasticity, loss of skin elasticity, increased skin wrinkles, sagging skin, slow skin barrier repair, rough skin, etc.

In some embodiments, the administration method of ergothioneine comprises in vivo and/or in vitro. When administered in vitro, for example, when smearing or applying externally, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered in vivo, such as by oral administration, the concentration of ergothioneine when administered can be 0.0001 wt %-20 wt %, such as 0.0001 wt %, 0.0002 wt %, 0.0003 wt %, 0.0004 wt %, 0.0005 wt %, 0.0006 wt %, 0.0007 wt %, 0.0008 wt %, 0.0009 wt %, 0.001 wt %, 0.002 wt %, 0.003 wt %, 0.004 wt %, 0.005 wt %, 0.006 wt %, 0.007 wt %, 0.008 wt %, 0.009 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt % etc., and any range between these values; when administered by injection, the concentration of ergothioneine can be 10 μg/L-50 g/L, such as 10 μg/L, 100 μg/L, 1000 μg/L, 1 g/L, 10 g/L, 20 g/L, 30 g/L, 40 g/L, or 50 g/L etc., and any range between these values.

In the use in preventing or alleviating skin issues associated with the reduction of type III collagen, preparing a product for preventing or alleviating skin issues associated with the reduction of type III collagen, or method for preventing or alleviating skin issues associated with the reduction of type III collagen of the present application, ergothioneine can be administered to the skin directly or indirectly, for example, by smearing, applying externally, injection or oral administration.

In the use of preparing a product for preventing or alleviating skin issues associated with reduction of type III collagen in the present application, the product may be a cosmetic, a medical device, a medicine, a food, etc. For example, ergothioneine can be formulated alone or in combination with other active ingredients into cosmetics, medical devices, medicines, foods, etc., for preventing or alleviating skin issues associated with the reduction of type III collagen.

The ergothioneine in the present application is for therapeutic or non-therapeutic purposes to achieve the above effects. For example, the role of ergothioneine in this application is fulfilled only for cosmetic purposes.

### EXAMPLE

The relevant information of the experimental materials and experimental equipments used in the examples is shown in Table 1.

TABLE 1

| Experimental materials and | |
|---|---|
| experimental equipments | Sources |
| Primary human dermal fibroblasts | Lifeline Company |
| Human fibroblast HFF | Shanghai Fuheng |
| | Biotechnology Co., Ltd. |
| DMEM medium, | Thermo |
| fetal bovine serum, | |
| 96-well | |
| cell culture plate | |
| CCK reagent | Tongren Biology |
| Human Collagen Type III ELISA kit | BIO-SWAMP Company |
| Cell culture incubator | Thermofisher i160 |
| Inverted microscope | Leica DMI1 |
| Biosafety cabinet | Labconco Class II |
| Microplate reader | Bio Tek 800TS |

### Example 1

### Experimental Steps:

### 1.1. Cell Plating

Human fibroblast (HFF) in logarithmic growth phase were collected, discarding the culture medium, washing them once with PBS, adding 0.25 wt % trypsin, incubating in a cell culture incubator for 1 minute, and then terminating digestion of the culture medium. The culture medium was centrifuged at 1000 rpm for 3 minutes, and the cells were resuspended in complete DMEM medium and counted. Human fibroblast (HFF) were inoculated into 12-well plates at a density of 3×10⁵ cells/ml, with 1 ml/well. After incubation in an incubator at 37° C. and 5% CO₂ for 24 hours, the culture medium was removed and the cells were washed with PBS.

### 1.2. Cell Grouping

The cells washed in step 1 were divided into two groups, namely a blank control group and an ergothioneine group, with 3 replicates in each group. Among them, the blank control group: 1 ml of serum-free culture medium; the ergothioneine group: 1 ml of serum-free culture medium containing 0.2 wt % ergothioneine.

After thoroughly mixing, cells in the above groups were placed in an incubator and cultured for another 72 hours.

### 1.3. Detection

Phase contrast microscopy was used to observe cell growth, morphology, and integrity of the cell monolayer, and changes in cell morphology and growth were recorded. The content of type III collagen was determined by using the human collagen III ELISA kit.

Type III collagen promotion rate was calculated as:

$$\frac{( Ergothioneine group - Blank control group )}{Blank control group} \times 100 \%$$

### Experimental Results:

As shown in Table 2, compared with the blank control group, the promotion rate of type III collagen in the ergothioneine group is significantly increased, indicating that ergothioneine can significantly promote the generation of type III collagen.

TABLE 2

| | Relative expression | Type III |
|---|---|---|
| | level of type | collagen promotion |
| | III collagen (%) | rate (%) |
| Blank control | 100.0 ± 12.3 | / |
| group | | |
| Ergothioneine | 148.3 ± 20.1** | 48.3 ± 20.1 |
| group | | |

Note: * represents P < 0.05,
** represents P < 0.01,
*** represents P < 0.001.

### Example 2

### 2.1. Sample Preparation

A sample solution was prepared by dissolving ergothioneine solid powder in a basic culture medium, and the concentration was adjusted to 0.001 wt %.

### 2.2. Testing of Type III Collagen Generation in Primary Human Dermal Fibroblasts

When the in vitro cultured fibroblasts reached 80-90% confluence, they were digested with trypsin and inoculated into 96-well plates. After 48 hours of monolayer culture in a 96-well plate, cells were treated with the ergothioneine sample prepared in step (1) (N=3), with a treatment group comprising 100 ng/mL IGF-1 was set as the positive control. After 48 hours, the cell culture supernatant was taken for ELISA assay of type III collagen, and the type III collagen content of the measured sample group was statistically compared with that of the blank control group to evaluate the effect of ergothioneine on the generation of type III collagen.

### 2.3. Statistical Methods

All results were presented as Mean±SD, and differences in measured data were compared using an unpaired t-test. P-value<0.05 is considered statistically significant.

### 2.4. Experimental Results

The effect of ergothioneine on the synthesis of type III collagen in dermal fibroblasts was tested using Human Collagen Type III ELISA kit, and the statistical results are shown in Table 3. 100 ng/mL IGF-1 in the positive control sample significantly promotes the synthesis of type III collagen in fibroblasts (P<0.05). Ergothioneine in the sample group significantly promotes the generation of type III collagen at a concentration of 0.001% (P<0.05).

TABLE 3

| | Blank control | Positive | Sample |
|---|---|---|---|
| Grouping | group | control group | group |
| Relative expression | 100.00% | 129.3% | 117.8% |
| level of type III | | | |
| collagen | | | |
| Standard Deviation (SD) | 0.08 | 0.05 | 0.07 |
| P value (compared to | / | 0.0103 | 0.0360 |
| the blank control | | | |
| group) | | | |

1. A collagen generation promoter, comprising ergothioneine as an effective ingredient.

2. The collagen generation promoter according to claim 1, wherein the collagen comprises type III collagen.

3. The collagen generation promoter according to claim 1, wherein its administration method comprises in vivo and/or in vitro.

4. Use of ergothioneine in promoting the generation of type III collagen.

5. The use according to claim 4, wherein the administration method of the ergothioneine comprises in vivo and/or in vitro.

6. Use of ergothioneine in preparing a product for promoting the generation of type III collagen in skin.

7. Use of ergothioneine in preparing a product for preventing or alleviating skin issues associated with the reduction of type III collagen.

8. The use according to claim 7, wherein the product comprises medicine, medical device, cosmetic or food.

9. A method for preventing or alleviating skin issues associated with the reduction of type III collagen, comprising administering ergothioneine to a subject in need thereof.

10. Use of ergothioneine in preventing or alleviating skin issues associated with the reduction of type III collagen.
